(19) United States
(12) Patent Application Publication
(10) Pub. No.: US 2024/0284986 A1
(43) Pub. Date: Aug. 29, 2024

(54) Vapour Generating Systems

(71) Applicant: JT International SA, Geneva (CH)

(72) Inventors: Jian Cheng Luo, Le Grand-Saconnex (CH); Layth Sliman Bouchuiguir, Bellevue (CH); Peter Smith, Leicester (GB)

(73) Assignee: JT International SA, Geneva (CH)

(21) Appl. No.: 18/686,741

(22) PCT Filed: Aug. 16, 2022
(86) PCT No.: PCT/EP2022/072841

§ 371 Date: Feb. 26, 2024

(30) Foreign Application Priority Data
Aug. 27, 2021 (EP) 21193571.3

(51) Int. Cl. A24F 40/53; A24F 7/00; A24F 40/40; A61B 5/1171; G06F 21/31

(52) U.S. Cl. CPC A24F 40/53; A24F 7/00; A24F 40/40; A61B 5/1171; G06F 21/316

(57) ABSTRACT

An apparatus and method for authenticating a user of a vapour generating system include emitting a transmitted signal from a mouthpiece of the system into an oral cavity of the user and detecting a received signal that includes reflections of the transmitted signal from the tongue of the user. The received signal is analysed to derive characteristics of it that include Doppler shifts resulting from the tongue moving towards or away from the receiver. Operation of the system is enabled only if the user moves their tongue in a predetermined pattern, for example by inaudibly speaking a password, so that the derived characteristics match corresponding characteristics of the authorized user that have been stored in a preliminary training step. The predetermined pattern of movement of the tongue is not easy for an unauthorized person to detect or to reproduce. The transmitted signal may be a high frequency acoustic signal.

### TECHNICAL FIELD

The present disclosure relates generally to vapour generating systems configured to heat a liquid to generate a vapour which cools and condenses to form an aerosol for inhalation by a user of the system. More specifically, it relates to authenticating the identity of users before they use such vapour generating systems.

### TECHNICAL BACKGROUND

The term vapour generating system (or more commonly electronic cigarette or e-cigarette) refers to handheld electronic apparatus that is intended to simulate the feeling or experience of smoking tobacco in a traditional cigarette. Electronic cigarettes work by heating a vapour generating liquid to generate a vapour that cools and condenses to form an aerosol which is then inhaled by the user. Accordingly, using e-cigarettes is also sometimes referred to as “vaping”. The vapour generating liquid usually comprises nicotine, propylene glycol, glycerine and flavourings.

Typical e-cigarette vaporizing units, i.e. systems or sub-systems for vaporizing the vapour generating liquid, utilize a heating element to produce vapour from liquid stored in a capsule, tank or reservoir. When a user operates the e-cigarette, liquid from the reservoir is transported through a liquid transfer element, e.g. a cotton wick or a porous ceramic block, and is heated by the heating element to produce a vapour, which cools and condenses to form an aerosol that can be inhaled. To facilitate the ease of use of e-cigarettes, removable cartridges are often employed. These cartridges are often configured as “cartomizers”, which means an integrated component comprising a liquid store, a liquid transfer element and a heater. Electrical connectors may also be provided to establish an electrical connection between the heating element and a power source. Such cartridges may be disposable, i.e. not intended to be capable of reuse after the supply of liquid in the reservoir has been exhausted. Alternatively, they may be reusable, being provided with means allowing the reservoir to be refilled with a new supply of vapour generating liquid.

A cartridge for an e-cigarette typically comprises an air inlet at a first end and an air outlet at a second, opposite end. (Considered from the viewpoint of a user of the system, the first end of the cartridge may also be termed the distal end and the second end of the cartridge may also be termed the proximal end or mouth end.) The first end of the cartridge is configured to be releasably connected to a main body of the vapour generating system, which may, for example, contain a power source and control electronics. A user inhales through a mouthpiece at the second end of the cartridge to draw air along an airflow path from the air inlet to the air outlet. The airflow path passes through a vaporization chamber, where liquid vaporized by the heating element mixes with the air.

There is a desire that individual vapour generating systems should be protected against use by unauthorized persons. This prevents infection or cross-contamination between different users. It also prevents the use of the vapour generating system by unsuitable persons such as children. Therefore there is a need for the vapour generating system to be able to authenticate a user prior to operation. In the event that the authentication process cannot classify the current user as an authorized user, the vapour generating system will be disabled from operating.

Hand-held vapour-generating systems are small in size and limited in battery power and, despite being reusable, they often have a short lifespan. Therefore the authentication process should not make heavy demands on the battery and should not require expensive sensors or other components to execute.

### SUMMARY OF THE INVENTION

The invention provides a vapour generating system, comprising:

a mouthpiece, the mouthpiece comprising a transmitter for emitting a transmitted signal into the oral cavity of a user and a receiver for detecting a received signal that includes reflections of the transmitted signal; and an analyser for analysing characteristics of the received signal to authenticate a user of the system, wherein the analysed characteristics include Doppler shifts in the reflections of the transmitted signal, and wherein the authentication is based at least in part on whether a pattern of the Doppler shifts corresponds to a predetermined pattern of movement of the tongue of the user.

The invention further provides a method of authenticating a user of a vapour generating system, comprising:

inserting a mouthpiece of the system into an oral cavity of the user:
emitting a transmitted signal from the mouthpiece of the system:
detecting a received signal that includes reflections of the transmitted signal; analysing the received signal to derive characteristics of the received signal that include Doppler shifts in the reflections of the transmitted signal; and authenticating the user based at least in part on whether a pattern of the Doppler shifts corresponds to a predetermined pattern of movement of the tongue of the user.

A vapour generating system naturally includes a mouthpiece that is received in the oral cavity of the user, in order for them to inhale the generated vapour. This makes it possible to authenticate the user by inviting them to perform a predetermined pattern of tongue movements, which can be detected by their effects on a signal transmitted and received by the mouthpiece. An advantage is that the tongue movements are hidden from unauthorized persons who may be nearby so such persons cannot learn the predetermined pattern and use it to enable operation of the vapour generating system. Even if unauthorized persons were to learn the predetermined pattern at a general level, it is highly unlikely that they would be able to reproduce the details of the way the authorized user moves their tongue, which are captured in the derived characteristics. Therefore the invention provides a secure apparatus and method for user authentication.

The derived characteristics of the received signal include a pattern of Doppler shifts of the transmitted signal when the user moves their tongue towards or away from the receiver. Characteristics based on such Doppler profiles have been demonstrated to be capable of distinguishing reliably between different users, at least in the analogous situation of lip movements. They provide a relatively simple method of deriving characteristics of the movements of the tongue by determining only the frequencies present in the received signal, which can be obtained by well-established signal processing techniques. This approach avoids the need for more complex sensors that are capable of determining an absolute value for the position of the tongue or the shape of the oral cavity.

The predetermined pattern preferably results from the user audibly or inaudibly speaking one or more predetermined passwords. This is a convenient way for the user to move their tongue in a rapid, repeatable and potentially quite complex manner, without the need to think consciously about the details of the movement.

The step of analysing the received signal may comprise converting the signal into the frequency domain then analysing signal gradients in the frequency domain. Well-established techniques such as Fast Fourier Transform may be used to convert the signal. Working in the frequency domain provides a convenient way to distinguish Doppler-shifted frequencies that are due to movements of the tongue from the principal frequency of the transmitted signal, as well as changes in those frequencies over time while the predetermined pattern of movement is being executed.

The step of authenticating the user may comprise applying a binary user classification test to assess features of the derived characteristics that can distinguish the user from other known users. A binary test is appropriate at least in the situation where a single user is authorized to use the vapour generating device. In that case, the authentication process only needs to find an answer “Yes” or “No” to the question, “Is the current user the authorized user of the current device?” instead of finding an identity for the user among all the possible known users of such systems. A suitable binary test can provide that answer in one or a few steps, without the need for extensive processing power or for the system to store details of all the known users.

The method of authenticating the user preferably comprises a preliminary user classification training step, which includes:
inserting the mouthpiece of the system into the oral cavity of an authorized user;
emitting the transmitted signal from the mouthpiece of the system;
detecting a received signal that includes reflections of the transmitted signal:

analysing the received signal to derive characteristics of the received signal that include a pattern of Doppler shifts in the reflections of the transmitted signal from the from the tongue of the authorized user when the authorized user moves their tongue in the predetermined pattern; and using a learning algorithm to compare the derived characteristics of the authorized user with a stored representation of corresponding characteristics of other known users and to generate the binary user classification test that can distinguish the authorized user from the other known users.

In this way the system is trained to recognize the authorized user so that it can prevent other, unauthorized users from operating the system in future. The step of analysing the received signal may be carried out while the user repeats the predetermined pattern of movements several times, in order to ensure that the generated binary test takes account of the variations in the user's execution of the movement.

The step of authenticating the user may further comprise applying a binary spoofer classification test to assess features of the derived characteristics that can distinguish the user from spoofers. Spoofers are unauthorized persons who may know the predetermined pattern of tongue movements and try to reproduce it to gain access to the vapour generating system. However, spoofers are unlikely to be able to replicate all the details of those movements as they are executed by the authorized user and therefore, according to this aspect of the invention, a further binary test is capable of distinguishing between the authorized user and such spoofers.

In certain embodiments of the invention, the transmitted and received signals are acoustic signals. Such signals are easily generated by a speaker and detected by a microphone, which are simple components with low power consumption that can be provided at small size and low cost in the mouthpiece of a vapour generating system. Alternative embodiments of the invention could be based on transmitted and received signals in the electromagnetic spectrum, for example microwaves similar to those used in vehicle speed detectors.

The transmitted acoustic signal preferably comprises a principal frequency that is higher than the normal range of human hearing, for example in the range 15 kHz to 30 KHz. An acoustic signal in that range will be transmitted effectively through the air in the oral cavity and will reflect effectively from the tongue, without being audible to or otherwise disturbing or harming the user.

In this specification, “authentication” is the process of determining whether a current user of the vapour generating system is an authorized user of the system. The outcome of authentication may accordingly be positive or negative.

Terms such as “upper” and “lower” denote the usual orientation of the vapour generating system during authentication or use of the system. It is understood that the system may be manufactured, transported, stored or sold in any orientation while remaining within the scope of the claimed invention.

### DESCRIPTION OF THE DRAWINGS

FIG. 1 is a schematic diagram of a vapour generating system according to the prior art, of a kind with which the present invention may be used.

FIG. 2 is a vertical cross section through the oral cavity of a user, schematically illustrating the use of a vapour generating system in accordance with the present invention.

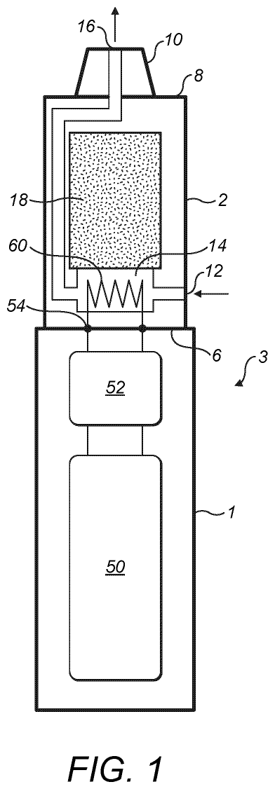

FIG. 1 schematically shows one possible configuration of a vapour generating system 3 in accordance with the present invention. A body 1 of the vapour generating system 3 houses a power source 50, which provides power to a control circuit 52. The vapour generating system 3 further comprises a cartridge 2, which houses a reservoir 18 containing a supply of a vapour generating liquid. A distal end 6 of the cartridge 2 is releasably connected to the body 1 so that, when the supply of vapour generating liquid in the reservoir 18 has been exhausted, the cartridge 2 can be removed and either replaced or refilled. At the proximal end 8 of the cartridge 2 there is a mouthpiece 10, which may be attached to or integral with the cartridge 2. An airflow path extends from an air inlet 12, through a vaporization chamber 14 to an air outlet 16 in the mouthpiece 10, whereby a user can inhale through the mouthpiece 10 to draw air along the airflow path through the vaporization chamber 14.

Means (not shown in FIG. 1) such as a cotton wick or a permeable ceramic block allows vapour generating liquid from the reservoir 18 to permeate slowly into the vaporization chamber 14. Terminals 54 couple the power source 50, via the control circuit 52, to a heater 60 in the cartridge 2. By applying power to the heater 60, the temperature in the vaporization chamber 14 can be increased, thereby causing the vapour generating liquid exposed in the chamber 14 to be vaporized. The resulting vapour becomes entrained in the flow of air drawn along the airflow path by the inhalation of the user. As the vapour is carried away from the vaporization chamber 14 towards the air outlet 16, it cools to a temperature suitable for inhalation and it may at least partly condense into droplets, of which the majority remain entrained in the airflow to be inhaled by the user.

Although FIG. 1 shows the cartridge 2 and body 1 of the vapour generating system 3 connected in an end-to-end configuration, it will be understood that in alternative embodiments of the invention the cartridge 2 could be releasably received inside the body 1. In that case, the mouthpiece 10 could be attached to or integral with the body 1 of the vapour generating system rather than the cartridge 2.

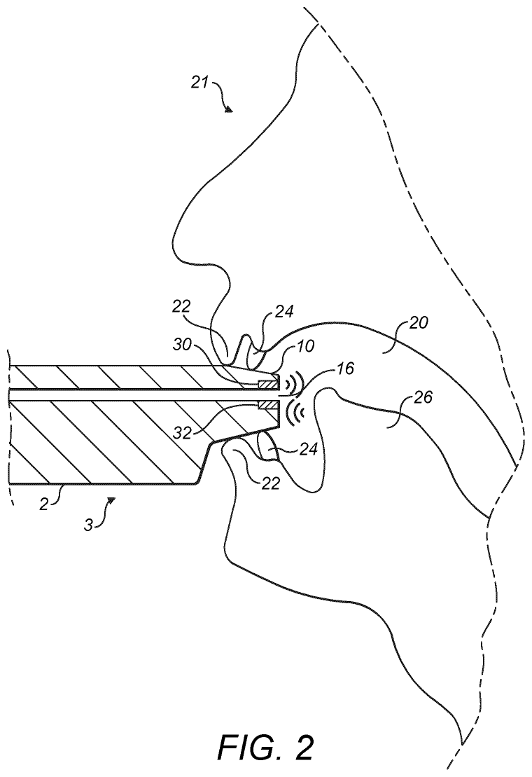

FIG. 2 schematically illustrates a vapour generating system of the general kind shown in FIG. 1, the mouthpiece 10 of the vapour generating system being received, via the mouth, in the oral cavity 20 of a user 21. Preferably the upper and lower lips 22 and the upper and lower teeth 24 of the user 21 rest on the exterior of the mouthpiece. Thereby, the air outlet 16 of the vapour generating system is located within the oral cavity 20, behind the teeth 24, such that its opening is generally directed towards the tongue 26.

In some embodiments of the invention, it may be important that the vapour generating system should be received in the mouth in a consistent position relative to the oral cavity. The shape or surface decoration (not shown) of the vapour generating system may allow the user to easily identify the correct orientation about the longitudinal axis in which to hold it. The mouthpiece 10 may be shaped with a recess, stop or other feature (not illustrated) to engage with the teeth 24 or lips 22 of the user 21 so that the vapour generating system can be positioned consistently in the longitudinal direction. In other embodiments, signal processing (described below) may be capable of allowing for different insertion positions of the vapour generating system.

The mouthpiece 10 comprises a transmitter 30 for emitting a signal into the oral cavity 20 and a receiver 32 for detecting a signal that includes reflections of the transmitted signal from surfaces within the oral cavity 20, particularly from the tongue 26. The transmitter 30 and the receiver 32 should be provided in the mouthpiece 10 at positions such that the line of sight between the transmitter 30 or receiver 32 and the relevant parts of the oral cavity 20 is unobstructed. In the schematic example of FIG. 2, they are positioned on a proximal end face of the mouthpiece 10. They could alternatively be positioned just inside the air outlet 16 to provide some protection from contact with the teeth 24 or tongue 26 during use and from the general environment when not in use. In the schematic example of FIG. 2, the transmitter 30 is positioned just above the air outlet 16 and the receiver 32 is positioned just below the air outlet 16 but they could alternatively be positioned on opposite (left and right) sides of the air outlet 16. In other embodiments the transmitter 30 and the receiver 32 could be located adjacent to one another. Some types of transmitter and receiver are available packaged together on a single microchip, e.g. the BGT60LTR11AIP microwave radar motion sensor chip available from Infineon®. For some applications it may be possible to use a single element as both the transmitter and the receiver.

The control circuit 52 of the vapour generating system 1 comprises an analyser for analysing the signal received by the receiver 32 and for authenticating the user 21 of the system based on characteristics of the received signal. The analyser may be implemented in a dedicated processor or in a general-purpose computer. The vapour generating system 1 may further comprise WiFi, Bluetooth® or other wired or wireless communication means (not illustrated), by which it may export data representing the received signal to another device such as a smart phone to carry out at least part of the analysis remotely, and by which it may import the result of the analysis in order to complete the authentication of the user or to act on the outcome of the authentication. In some embodiments of the invention, the characteristics determined by the analyser may be compared against a database of characteristics of known users in order to identify the current user; and it may be convenient for that database to be stored in a central location remote from the vapour generating system 3 for ease of updating. In most applications of the invention, a single permitted user is associated with a particular vapour generating system. The system therefore does not need to identify the current user among all possible users but only to carry out a binary test (giving a “yes” or “no” answer) to determine whether the detected characteristics of the current user match the stored characteristics of the permitted user.

To use the system 3, the user first activates it. This may be done in a number of ways, for example by pressing a button or blowing into the mouthpiece 10. The system 3 may comprise a movement sensor that causes it to activate when picked up by the user or a pressure sensor or light sensor (not illustrated) may respond to the mouthpiece 10 being placed in the user's mouth.

Once activated, the vapour generating system 3 needs to authenticate the user before it can be operated. Typically, until the user has been authenticated, the control circuit 52 will not supply power to the heater 60 so the system 3 cannot generate any vapour. The system 3 may indicate to the user that authentication is required, e.g. by emitting a sound or by vibrating. In accordance with the invention, the authentication is carried out by the user moving their tongue 26 according to a predetermined pattern that is characteristic of the user. The chosen pattern is not necessarily unique among all the users of such systems because two users implementing nominally the same pattern are likely to do so in ways that are sufficiently different to be distinguished by the analyser.

The predetermined pattern may be a simple sequence of moves: for example, using the tip of the tongue 26 to touch the left teeth 24 once, then the right teeth 24 twice, followed by the palate. Preferably, the predetermined pattern results from the movement of the tongue as the user speaks one or more predetermined passwords. Provided the passwords comprise a sufficient number of syllables, such a pattern is likely to be more complex than a simple sequence and also more consistent because the user can speak the passwords quickly and without consciously thinking about the movements involved. Of course, the enunciation of the passwords will be affected by the presence of the mouthpiece 10 between the user's teeth so it may be easier for the user to avoid words containing elements that are difficult to enunciate in those circumstances, such as the letters “p” and “b”. On the other hand, the difficult of enunciation does not matter to the system, which only needs to be able to detect a consistent pattern of movements rather than well-enunciated words. The passwords are preferably spoken silently or inaudibly so that they may not become known to other persons in the vicinity—although, even if another person knew the passwords, it is considered unlikely that they would be able to reproduce the user's characteristic tongue movements while speaking them. Because such a system does not depend on sounds spoken by the user, it will work equally well in noisy environments.

The transmitter 30 emits a signal and the receiver 32 detects movements of the tongue 26 through changes in the signal that is reflected from the tongue. The received signal is processed to detect Doppler shifts in the frequency of the signal when it is reflected from the moving tongue 26. As the user moves their tongue 26 in the predetermined pattern and the tongue moves towards and away from the receiver 32, the Doppler shifts will change over time in a characteristic way. With this technique, it is not necessary to obtain direct measurements of the position or the speed of movement of the tongue: it is sufficient to work only with those Doppler profiles. In order that small Doppler shifts can be detected, the transmitted signal may be a substantially continuous tone that is emitted at a single principal frequency or in a narrow band around the principal frequency.

In some embodiments of the invention, the transmitter 30 may be a speaker that emits a high frequency acoustic signal and the receiver 32 may be a microphone that is sensitive to similar frequencies. In this context, “high frequency” means a frequency that is above the normal range of human hearing, for example in the range 15 kHz to 30 kHz, whereby the tone is not distracting or annoying to the user. The frequency should be one that is capable of transmission through air with low attenuation, unlike the even higher frequencies (>1 MHz) used in medical ultrasound imaging, which can only be transmitted successfully through solid tissue.

A similar technique based on high frequency acoustic signals has been demonstrated in the research paper “LipPass: Lip Reading-based User Authentication on Smartphones Leveraging Acoustic Signals” delivered by Li Lu et al. at the 2018 IEEE Conference on Computer Communications (DOI: 10.1109/INFOCOM.2018.8486283). In that demonstration, acoustic signals at e.g. 20 kHz were directed towards a user's mouth and reflected signals were analysed to extract unique characteristics of the movement of the lips while the user spoke predetermined passwords. A Fast Fourier Transform was used to convert the sampled amplitudes detected by a microphone into the frequency domain, thereby generating a profile of how the frequencies of the reflected signal change over time because of the changing Doppler effect of the moving lips. The Doppler profiles of acoustic signals reflected from users' lips while speaking indicated that there are unique lip movement patterns for different users, even when speaking the same words. Thus, when the same user speaks the words on different occasions, the resulting profiles share common characteristics, which are not present when other users speak the same words.

To characterize the lip movements, the LipPass demonstration used a deep-learning-based method to extract efficient features from the Doppler profiles. The method then employed Support Vector Machine and Support Vector Domain Description techniques to construct binary classifiers and spoofer detectors for user identification and spoofer detection, respectively. Thereafter, a binary tree-based authentication approach was employed to accurately authenticate each user, leveraging the binary classifiers with respect to registered users and spoofer detectors with respect to spoofers, who are not registered users but are other persons who might try to imitate the manner in which the permitted user speaks the passwords.

The same techniques may be used in systems according to the present invention to derive characteristics from the Doppler profiles by which the user can be authenticated. As an alternative to acoustic signals, the vapour generating system 3 may be provided with a transmitter 30 and a receiver 32 that operate using radio frequency electromagnetic radiation, for example in the frequency range above 1 GHz. Just as in the acoustic case, movements of the tongue towards or away from the receiver 32 will cause Doppler shifts in the frequency of the reflected signal, which can be detected by the receiver 32 and used to generate a Doppler profile of the changing frequency shifts over time. The Doppler profile of a user moving their tongue 26 in a predetermined pattern, such as by audibly or inaudibly speaking a password, comprises characteristics by which they can be distinguished from other users and from spoofers, and the system 3 can therefore use the detected Doppler profile as a way of authenticating the user.

If the authentication process judges a user to be authorized to use the system 3, then features of the system including the heater 60 will be enabled to operate in a conventional manner. The operation may be permitted to continue to be enabled for a predetermined time or until a condition such as the end of a smoking session is determined to have been reached.

Before the first use of the system 3, a preliminary user classification training process will need to be carried out to train the system to recognize the authorized user. The system 3 is activated and the authorized user places the mouthpiece 10 in their mouth. The system 3 indicates to the user that they should move their tongue 26 in their chosen predetermined pattern, for example by speaking one or more passwords. While the authorized user does that, the transmitter 30 emits a signal, the receiver 32 detects signals reflected from the moving tongue 26 and the analyser processes the detected signals to generate a Doppler profile in the same manner as during the authentication process. However, during the training process the Doppler profile is analysed to derive characteristics of it that may be used to distinguish the current, authorized user from other unauthorized users. The system 3 may issue a warning to the authorized user if suitable characteristics cannot be derived from the Doppler profile, for example if the password is not long enough to comprise characteristics that can uniquely identify the authorized user.

Preferably, the system 3 prompts the authorized user to repeat the pattern of tongue movements a number of times, in order that variations in successive repetitions of the movements can be allowed for. The number of repetitions may be predetermined or the repetitions may be continued until sufficient data has been collected. The successive Doppler profiles may be stored in a memory of the system 3 and then deep learning techniques such as Support Vector Machine and Support Vector Domain Description may be applied to them to construct binary classifiers for user identification and spoofer detection specific to the authorized user. Construction of the binary classifiers may be carried out by the analyser within the control circuit of the system 3 or, if the system 3 is provided with means for external communication, the construction step may be exported for processing at a remote location. The system 3 may issue a warning to the authorized user if suitable classifiers cannot be constructed, for example because there is too much variation between the repeated tongue movements.

The binary classifiers are stored in the memory of the vapour generating system 3 to be used during authentication of the user by the system 3 on subsequent occasions. The binary classifiers may also be stored at a remote location so that if the authorized user wishes to use a new vapour generating system 3 of the same kind, the stored binary classifiers may be downloaded from the remote location to the new system 3 as an alternative to the authorized user working through a new training process to create new classifiers.

1. A vapour generating system, comprising:
a mouthpiece, the mouthpiece comprising a transmitter for emitting a transmitted signal into an oral cavity of a user and a receiver for detecting a received signal that includes reflections of the transmitted signal; and
an analyser for analysing characteristics of the received signal to authenticate a user of the system, wherein the analysed characteristics include Doppler shifts in the reflections of the transmitted signal, and wherein the authentication is based at least in part on whether a pattern of the Doppler shifts corresponds to a predetermined pattern of movement of a tongue of the user.

2. The vapour generating system according to claim 1, wherein the transmitted and received signals are acoustic signals.

3. The vapour generating system according to claim 2, wherein the transmitted signal comprises a principal frequency that is higher than the normal range of human hearing.

4. The vapour generating system according to claim 2, wherein the transmitted signal comprises a principal frequency in the range of 15 kHz to 30 kHz.

5. A method of authenticating a user of a vapour generating system, comprising:
inserting a mouthpiece of the system into an oral cavity of the user;
emitting a transmitted signal from the mouthpiece of the system;
detecting a received signal that includes reflections of the transmitted signal;
analysing the received signal to derive characteristics of the received signal that include Doppler shifts in the reflections of the transmitted signal; and
authenticating the user based at least in part on whether a pattern of the Doppler shifts corresponds to a predetermined pattern of movement of a tongue of the user.

6. The method according to claim 5, wherein the predetermined pattern results from the user audibly or inaudibly speaking one or more predetermined passwords.

7. The method according to claim 5, wherein analysing the received signal comprises converting the signal into a frequency domain, then analysing signal gradients in the frequency domain.

8. The method according to claim 5, wherein authenticating the user comprises applying a binary user classification test to assess features of the derived characteristics that can distinguish the user from other known users.

9. The method according to claim 8, further comprising a preliminary user classification training step, which includes:
inserting the mouthpiece of the system into the oral cavity of an authorized user;
emitting the transmitted signal from the mouthpiece of the system;
detecting a received signal that includes reflections of the transmitted signal;
analysing the received signal to derive characteristics of the received signal that include a pattern of Doppler shifts in reflections of the transmitted signal from a tongue of the authorized user when the authorized user moves the tongue in the predetermined pattern; and
using a learning algorithm to compare the derived characteristics of the authorized user with a stored representation of corresponding characteristics of other known users and to generate the binary user classification test that can distinguish the authorized user from the other known users.

10. The method according to claim 5, wherein authenticating the user comprises applying a binary spoofer classification test to assess features of the derived characteristics that can distinguish the user from spoofers.

11. The method according to claim 5, wherein the transmitted and received signals are acoustic signals.

12. The method according to claim 11, wherein the transmitted signal comprises a principal frequency that is higher than the normal range of human hearing.

13. The method according to claim 11, wherein the transmitted signal comprises a principal frequency in the range of 15 kHz to 30 KHz.

14. The vapour generating system according to claim 1, wherein the analyser comprises a processor configured to authenticate a user of the vapour generating system, the authenticating comprising:
emitting a transmitted signal from a mouthpiece of the system inserted into an oral cavity of the user;
detecting a received signal that includes reflections of the transmitted signal;
analysing the received signal to derive characteristics of the received signal that include Doppler shifts in the reflections of the transmitted signal; and
authenticating the user based at least in part on whether a pattern of the Doppler shifts corresponds to a predetermined pattern of movement of a tongue of the user.
